(19) United States
(12) Patent Application Publication
(10) Pub. No.: US 2021/0251886 A1
(43) Pub. Date: Aug. 19, 2021

(54) ORAL MUCOSAL DELIVERY SYSTEMS COMPRISING MONOPHASIC CONCENTRATE OF TERIPARATIDE

(71) Applicant: ZIM LABORATORIES LIMITED, Nagpur, Maharashtra (IN)

(72) Inventors: Anwar Siraj DAUD, Nagpur (IN); Nidhi Prakash SAPKAL, Nagpur (IN); Minal Nandkumar BONDE, Nagpur (IN); Padma Venkitachalam DEVARAJAN, Mumbai (IN); Darsheen Jitendrabhai KOTAK, Mumbai (IN)

(73) Assignee: ZIM LABORATORIES LIMITED, Nagpur, Maharashtra (IN)

(21) Appl. No.: 16/973,747

(22) PCT Filed: Jun. 10, 2019
(86) PCT No.: PCT/IN2019/050444

§ 371 Date: Dec. 9, 2020

(30) Foreign Application Priority Data
Jun. 10, 2018 (IN) 201821021663

(51) Int. Cl. A61K 9/00; A61K 38/29; A61K 9/08; A61K 47/10; A61K 47/20; A61K 9/20

(52) U.S. Cl. CPC A61K 9/006; A61K 38/29; A61K 9/20; A61K 47/10; A61K 47/20; A61K 9/08

(57) ABSTRACT

This invention comprises a water free liquid composition comprising Teriparatide for transmucosal delivery that adheres to mucosa after coming in contact with mucosal surface and a system of making dosage form from the same and delivering to mucous membrane at various locations though liquid drops, capsule or tablets. The liquid composition is characterized by being a monophasic composition. The liquid monophasic composition comprises a non-aqueous liquid as carrier, a penetration enhancer/permeation enhancer, stabilizer and a surfactant. The liquid composition of Teriparatide intended to be dispensed to a mucosal membrane of oral/buccal cavity as liquid drops, or of oral/buccal cavity or nasal cavity as an oral or nasal spray or of oral/buccal cavity as an oral film; or of a part of alimentary canal as incorporated in a capsule or a tablet as an ingredient.

### FIELD OF INVENTION

This invention pertains to oral mucosal delivery system comprising liquid monophasic of Teriparatide. The invention still more particularly comprises liquid monophasic formulation or liquid monophasic composition incorporated in suitable dosage forms of Teriparatide for sublingual or buccal delivery.

### BACKGROUND

Osteoporosis is a systemic skeletal disease with low bone mass and deterioration of bone tissue. Teriparatide is a peptide used for the treatment of osteoporosis, a condition that afflicts both men and women worldwide. There are about 200 million patients all over the world and out of which 36 million patients are in India. Teriparatide is the only available anabolic agent administered by a once a daily subcutaneous injection of 20 μg. The recommended duration of treatment is daily injections for about 24 months. A non-invasive method of administration of effective amount of Teriparatide is a long standing need.

Teriparatide is a peptide and is available in injectable form only. Being a large molecule its permeation through biological membrane is very poor. Further, Teriparatide is unstable in water but is stable at acidic pH. Further, it is administered as subcutaneous injection. Injection as a dosage form is not patient friendly. It is an invasive dosage form and needs skilled person for its administration. Osteoporosis occurs mostly to geriatric population and they are not in stage to learn self-administration of such product. There is great shortage of skilled persons in the developing countries especially in the rural area. With the mishandling or incorrect technique of administration of injectable dosage forms, fatalities may occur. Additionally, being sterile, this is expensive as compared to non-sterile products. Due to all these issues, injections are a very inconvenient dosage form and such products suffer from treatment non-compliance resulting in increased patient discomfort and healthcare cost. Additionally, it gets degraded by digestive system and, hence, has poor bioavailability when given by oral route. Therefore there is a need of non-invasive oral formulation that will offer a safe and efficacious option for Teriparatide treatment.

Oral transmucosal routes like sublingual, buccal are preferable as non-invasive routes instead of the conventional subcutaneous injection.

WO 2012113116 A1 has disclosed an emulsion (W/O) containing hydrophilic biomolecules, their preparation and use. The emulsion contains hydrophilic biological macromolecule, water phase, oil phase, emulsifying agent, vitamin E and/orester derivatives of vitamin E. The hydrophilic biomolecules is selected from proteins or polypeptides, polysaccharides, and nucleic acids. Use of ester derivatives of vitamin E or vitamin E itself as a carrier for a hydrophilic bio-macromolecules is disclosed, which is found to significantly improve the oral bioavailability. It claims administration by oral route but in the studies the microemulsion was directly injected into the rat intestine and a 23.13% bioavailability compared to subcutaneous injection is claimed. Both the tₘₐₓ and half-life are shown to be similar to subcutaneous injection. But since it was not administered by oral route therefore, it is not the indicative of true oral bioavailability. Both the tₘₐₓ and half-life are shown to be similar to subcutaneous injection.

Compositions for buccal delivery of parathyroid hormone are disclosed in (WO 2006076692 A1). This invention discloses buccal delivery of parathyroid hormone or a fragment or an analogue thereof (collectively, the “PTH component”) with a delivery agent, pharmaceutical compositions for buccal administration comprising a PTH component and a delivery agent, and methods of preventing or treating osteoporosis or stimulating new bone formation in an animal by buccally co-administering a PTH component and a delivery agent. 4-MOAC-(N2 hydroxy4methoxybenzoyl) amino caprylic acid, NAC-N(8[2hydroxybenzoyl]amino) caprylic acid—NAD and SNAD—N (8 [2 hydroxybenzoyl] amino) decanoic acid 5-CNAC—N(8[2hydroxy5chlorobenzoyl] amino) octanoic acid 4-CNAB-N(8[2hydroxy5chlorobenzoyl] amino) butanoic acid have been used as delivery agent. PTH (1-34) was administered to rats buccaly with and without a delivery agent. The buccal dosage of PTH used was 0.2 mg/kg body weight. The dose of delivery agent was 200 mg/kg body weight. In dogs about 3.3% bioavailability was found by buccal route and 4.8% by oral route.

Non-covalent soluble complexes of Teriparatide with polysaccharides and a dosage form of Teriparatide for oral administration is claimed in WO 2012130193 A1. The invention relates to the stabilization of Teriparatide by formation of soluble complexes with polysaccharides (β-glucan, chitosan, alginic acid, or their salts) enabling its oral administration. Beta glucan, chitosan and its salts and alginic acid and its salts/aqueous solution of Teriparatide is mixed with an aqueous solution of the polysaccharide and the mixture is incubated at 0-40° C. for 0.5 to 72 hrs, 50 mcg/kg dose gives 17% of bioavailability with such non-covalent complexes. The final composition can be made either in solid or in liquid form for oral delivery of Teriparatide. In this case, the invention was found to show tₘₐₓ and half-life comparable to subcutaneous injection. Bioavailability was very good in the presence of chitosan. Chitosan is soluble in acidic pH. However, this formulation involves lyophilisation as a processing step, which is an expensive technique and is not preferred for large scale production.

Stabilized Teriparatide Solutions are reported by U.S. Pat. No. 7,550,434 B2. A stabilized pharmaceutical composition in the form of a solution for parenteral administration of a parathyroid hormone is described, wherein the therapeutically active ingredient is stabilized with a buffer and a polyol. Preferred preparation contains in an aqueous solution human PTH (1-34), mannitol (3-10%), an acetate or tartrate buffering agent and m-cresol or benzyl alcohol as a preservative.

Stable pharmaceutical dosage forms of Teriparatide disclosed in US 2006/0189533 A1 is a dosage form of parathyroid hormone (1-34) (PTH) comprising an aqueous pharmaceutical formulation for aerosolized intranasal delivery of PTH having a maximum bioavailability of about 14.6%, wherein the formulation comprises a therapeutically effective amount of PTH and polysorbate, and wherein least 90% of the PTH can be recovered after storage for 24 weeks at 5.degree in the dose ranges from 200 μg to 1000 μg.

Enhanced mucosal delivery of Parathyroid hormone is also disclosed in US 2006/0052306/A. It comprises an aqueous pharmaceutical composition for intranasal delivery of PTH, comprising a PTH molecule, and one or more excipients selected from the group consisting of a chelating agent, an alcohol, and a surface active agent. The composition contains cyclodextin, DDPC, EDTA, Triton-X, Tween-80, sodium benzoate, sorbitol. The pH of formulation is 4. The relative bioavailability disclosed in both US 2006/0189533 A1 and US 2006/0052306/A is same.

Composition and methods for enhanced mucosal delivery of parathyroid hormone has been disclosed in U.S. Pat. No. 7,244,709 B2. This patent claims a formulation for delivery of PTH across a nasal mucosal cellular layer, comprising an aqueous mixture of PTH, a cyclodextran and didecanoylphosphatidylcholine at concentrations sufficient to enhance permeation across the cellular layer, wherein the formulation consists of droplets, of which less than 10% are less than 10 microns in diameter. In vivo PK study has been performed with 5 mcg/kg (Sub Cutaneous Injection) and 50 mcg/kg (IN) in rabbits. half-life is similar to injectable formulation and bioavailability is 7.3% as compared to Sub-Cutaneous injection. The above formulations for intranasal administration provide disadvantage of not being useful when patients suffer from common cold and rhinitis. They are also not patient friendly compared to sublingual and buccal formulations.

The first available brand of injectable Teriparatide is “Forted” by Eli Lilly that was launched in 2002. In 2008, Ranbaxy launched in India (Bonista-Teriparatide Injection). To date there is no formulation in the market for administration by transmucosal oral route which is the most preferred non-invasive, non-injectable route that is a viable alternative for injectable PTH; and that is a long standing need for those who are at risk or are having osteaoporosis. Objective of the instant invention was to fulfill that need.

### SUMMARY

This invention comprises a water free liquid composition comprising Teriparatide for transmucosal delivery that adheres to mucosa after coming in contact with mucosal surface and a system of making dosage form from the same and delivering to mucous membrane at various locations through dispensing the liquid drops, or dispensing capsule or tablets comprising the liquid composition as an ingredient.

The liquid composition is characterized by being a monophasic composition. The liquid monophasic composition comprises a non-aqueous liquid as carrier, a penetration enhancer/permeation enhancer, stabilizer and a surfactant. The function of a penetration enhancer/permeation enhancer, stabilizer and a surfactant may be performed by separate ingredients or an ingredient having more than one function may also be used for making the liquid monophasic composition. A liquid carrier may be selected from the list consisting of Propylene Glycol, Polyethylene Glycol 200, Polyethylene Glycol 400, Glycerol, Ethanol and functionally equivalent liquid other than above and mixture thereof. A penetration enhancer/permeation enhancer is selected form the list consisting of N-acetyl Cysteine, Propylene Glycol, TPGS (d-α-Tocopheryl polyethylene glycol 1000 succinate), Tween 20 and functionally equivalent penetration enhancer/permeation enhancer other than above, and mixture thereof. A stabilizer is selected from the list consisting of N-acetyl Cysteine, TPGS, or a stabilizer other than above, and mixture thereof. A surfactant is selected from the list consisting of TPGS, Tween 20 or a functionally equivalent surfactant other than above. The liquid composition of Teriparatide according to this invention is intended to be dispensed to a mucosal membrane of oral/buccal cavity as liquid drops, or of oral/buccal cavity or nasal cavity as an oral or nasal spray or of oral/buccal cavity as an oral film or tablet; or of a part of alimentary canal as incorporated in a capsule or a tablet as an ingredient. The capsule and tablet are intended or not intended for site specific delivery of the liquid composition.

### DETAILED DESCRIPTION OF THE INVENTION

### Brief Description of FIGURES and Legends

FIG. 1: Pharmacokinetic study of Teriparatide administered as subcutaneous injection and as a monophasic liquid composition comprising a penetration enhancer and a surfactant at dose of 50 μg and 100 μg/kg of Teriparatide.

### DETAILED DESCRIPTION OF THE INVENTION

This invention comprises a liquid composition comprising an Active Ingredient for transmucosal delivery of the Active Ingredient by transforming into a mucoadhesive gel after coming in contact with a mucous membrane. It was a surprising observation that when such a liquid composition comprising 0.1% Teriparatide was administered at 100 μg/kg to rabbits, that gave about 14% bioavailability when this liquid composition was administered as drops at 100 μg/kg dose at sublingual location in comparison to 10 μg/kg dose by subcutaneous injection and half-life(t_{1/2}) of 47±17.32, which is double of the half-life of 20.89±12.63 for subcutaneous injection. Thus, this composition gave viable alternative to injectable formulation. The injection has to be given one injection each day for over 24 months. The exposure of a patient for total period of half-life after administration of each dose and number of doses each day determines the efficacy and period of total treatment required to achieve desired effect. An injection cannot be given multiple number of times each day because of the reasons described earlier; whereas the liquid composition of the instant invention provides more than double period of hail-life per dose and offers the convenience of self-administration therefore, can be prescribed for administration for more than once a day. Thus, the composition of the instant invention is far more effective and far more convenient for administration.

A person skilled in the art of pharmaceuticals will immediately recognise that the same liquid composition can be used for efficient trans-mucosal delivery of a number of Active Ingredients other than Teriparatide also for drugs that can be accommodated in a volume of the liquid that is practical for the system of delivery of Active Ingredients of the instant invention. Thus scope of the instant invention extends to all such Active Pharmaceutical Ingredients that are obviously equivalent. A person skilled in the art may explore Active Pharmaceutical Ingredients other than Teriparatide, such as Vitamin B12, therapeutic peptides and proteins such as Insulin, calcitonin, GLP, octreotide, etc. and the like to employ full scope of this invention. Particularly, the Active Ingredients that are required in microgram quantities could be ideal subjects for administering through liquid composition of his invention.

The liquid composition comprises a monophasic concentrate of the Active ingredient. The term “monophasic” is used here to clarify that the liquid does not have more than one phase i.e. it does not contain an emulsion or micro-emulsion of any type or does not contain mixture of separate phases of liquids. It may, however, contain undissolved solid Active Ingredients which may still be absorbed via transmucosally through on account of the composition and due to muco adhesiveness of the resulting gel after coming in contact with the mucosa.

In one embodiment of this invention, the liquid composition of the instant invention comprises amonophasic mixture of a liquid as carrier, a penetration enhancer/permeation enhancer, stabilizer, and a surfactant. For the purpose of this specification, both the terms “Penetration enhancer” and “Permeation enhancer are used to denote same meaning and same scope. The monophasic mixture gels as soon as it comes in contact with water.

In one embodiment of this invention, the Active Ingredient used is Teriparatide. In the embodiment illustrating Teriparatide as Active Ingredient, Propylene Glycolis used as a liquid carrier although other functionally equivalent liquid can also be used in its place, N-acetyl Cysteineis used as a penetration enhancer/permeation enhancer, although or other ingredient functionally equivalent can also be used, TPGS (d-α-Tocopheryl polyethylene glycol 1000 succinate) is used as a surfactant although other ingredient functionally equivalent as a surfactant can replace the same. Some of the ingredients may have dual function, such as N-acetyl cysteine is a penetration enhancer as well as stabilizer.

The liquid composition can be applied directly as drops to intended portion of buccal cavity, which includes sub-lingual and inside surface of both cheeks, or as an oral or nasal spray to a mucosal membrane, as a sub-lingual film comprising the instant liquid as one of its ingredients and the like, or to mucous membrane of a part of alimentary canal as an ingredient incorporated in a capsule or a tablet that is intended or not intended for site specific delivery of the liquid composition.

This invention also comprises a process of making a liquid composition comprising an Active Ingredient for transmucosal delivery of the Active Ingredient by transforming into a mucoadhesive gel after coming in contact with a mucous membrane; and a system of using the liquid itself as a dosage form or incorporating the liquid into a dosage form for delivery at desired site in the body.

The process comprises steps of making two solutions, Solution A in the liquid carrier by solubilizing in it the penetration enhancer and the Active Ingredient; and Solution B in the liquid carrier of a surfactant; and mixing the two to make the liquid as a monophasic liquid carrying the Active Ingredient.

### Example 1: Preparation of a Water Free, Buffer Free, Monophasic Teriparatide Liquid Composition

| | Formulation |
|---|---|
| Ingredients | Quantity (%) |
| Teriparatide | 0.1 |
| N-acetyl Cysteine | 0.3 |
| Propylene Glycol | 40 |
| TPGS (d-α-Tocopheryl polyethylene | 59.4 |
| glycol 1000 succinate) | |
| Total | 100 |

### Procedure:

1) About half of the amount of total Propylene glycol (PG) required was weighed and N-Acetyl cysteine (NAC) was added in to it. It was allowed to solubilize completely.
2) Once the N-Acetyl Cysteine is solubilized completely in PG, accurately weighed amount of Teriparatide was added and it was solubilized completely (Solution A).
3) Accurately weighed amount of TPGS was mixed with the remaining half of the quantity of accurately weighed PG with gentle stirring until TPGS was mixed with PG to make a homogenous solution (Solution B).
Solution A and B were mixed with mild stirring to make a homogenous solution of a water free, buffer free, monophasic Teriparatide liquid composition.

### Example 2: Preparation of Sub-Lingual Film Using Monophasic Composition of Teriparatide

| Ingredients | Quantity in percent |
|---|---|
| HPMC 15 cps | 70.5 |
| Titanium Dioxide | 0.5 |
| Sucralose | 1 |
| Glycerol | 6 |
| Neusilin | 2 |
| Monophasiccomposition | 20 |
| Water | Quantity Sufficient |

An Orally dissolving thin film dosage from incorporating monophasic liquid composition was made by using following steps:
1. A dispersion was prepared by dissolving HPMC and Glycerol in required quantity of water. To this dispersion Titanium Dioxide, Sucralose and neusilin was added and mixed completely. This dispersion was allowed to stand for four hours.
2. The monophasic liquid concentrate made according to Example 1 was added to the solution prepared in step I and resulting dispersion was casted in films of desired thickness.
3. These films were then dried in a drying chamber to the desired level of moisture content.

### Example 3: Preparation of Freeze-Dried Tablet Using Monophasic Composition of Teriparatide

| Ingredients | Quantity in percent |
|---|---|
| HPMC 15 CPS | 25 |
| Mannitol | 53.45 |
| Titanium Dioxide | 0.55 |
| Sucralose | 1 |
| Monophasic concentrate | 20 |
| Water | Quantity Sufficient |

Freeze Dried Sublingual Tablets were Prepared by Following Steps:
1. HPMC E15, Mannitol, Sucralose and titanium dioxide were mixed thoroughly.
2. Powder blend prepared in step 1 was mixed with water until homogenous dispersion was formed.
3. Accurately weighed quantity of monophasic liquid composition prepared in example 1 was added to homogenous dispersion prepared in step 2 and mixed to form uniform dispersion.
4. A volume of 250 μl of above homogenous dispersion was filled in preformed Alu-alu blister and kept at −60° C. for 6-8 hrs in deep freezer. Frozen units were freeze dried using Labconco freeze-drying system (Free Zone 4.5, USA). Sublimation lasted for 12 h at a vacuum pressure of 10-50×10⁻³ bar, with the condenser surface temperature maintained at less than −50° C. to provide freeze dried tablets. Solid, circular, porous tablets are obtained that dissolve rapidly in the oral cavity.

### Example 4: Pharmacokinetic Studies in Rabbits

### Study Design:

Female New Zealand White Rabbits (2 Kgs) were divided into 3 groups with 3 animals in each group. Group I was administered subcutaneous injection of Teriparatide with a dose of 10 μg/kg while group II and II were administered sublingual Teriparatide monophasic liquid composition with a dose of 50 and 100 μg/kg respectively. Bloodsamples (0.5 ml) were collected from the ear vein of the rabbits at time intervals of 15, 30, 60, 90, 120, 180, 240 and 360 minutes post dosing into micro-centrifuge tube. Blood samples were allowed to clot and serum was collected by centrifugation at 3,000 rpm for 10 min. Serum Teriparatide concentration was measured using the rabbit ELISA Kit. Pharmacokinetic parameters were calculated using non-compartmental model analysis as shown in table 1

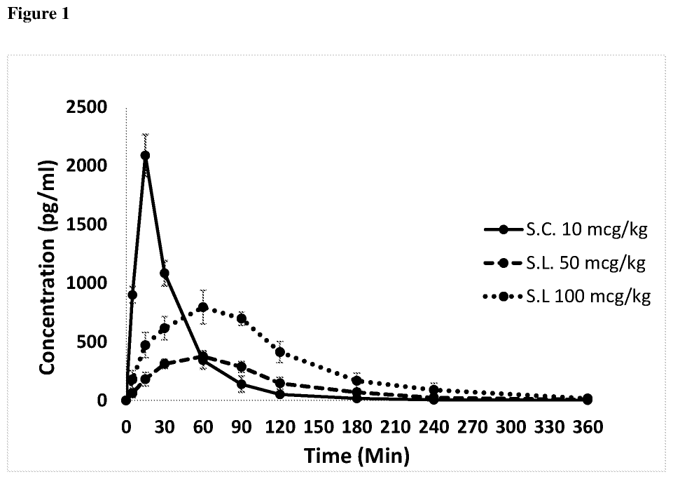

Thus, FIG. 1 and Tables 1 show that half-life of 100 μg/kg of sub-lingual application of the monophasic mixture of Teriparatide is 47.34±17.34, which is more than the half-life of sub-cutaneous injection. Thus, monophasic liquid composition of Teriparatide made as per Example 1 is a viable alternative for administration of Teriparatide over injectable; and more effective since its half-life is more than the injectables. It also opens up a possibility of giving more dosages per day so as to achieve uniform plasma concentration for longer period of the day; this is not possible with injectables since they cannot be administered several times a day nor can the quantity injected per dose can be increased.

In animal trials on rabbits, relative bioavailability was observed to be 100% for 10 μg/kg of subcutaneous injection, 5.47% for 50 μg/kg dose through sub-lingual film and 14.03% for 100 μg/kg dose through sub-lingual film. Thus, blood concentration of Teriparatide by subcutaneous injection and by 100 μg/kg sub-lingual film was comparable; and whereas half-life (t_{1/2}) of subcutaneous injection was 20.89±12.63 min, that of 100 μg/kg sub-lingual film was 47.34. Thus, this invention shall make it possible to achieve blood levels of Teriparatide as good as the one achieved with subcutaneous injection, and in addition, it shall have a half-life that is about twice as that. Since it is practically possible to increase the frequency of the dosages per day in case of sub-lingual administration of the monophasic liquid, this invention has provided a non-invasive and more patient friendly method of providing treatment of Teriparatide than the method based on injections, which is more efficacious to since it may be completed in shorter period too.

TABLE 1
Pharmacokinetic study ofTeriparatide administered as sub-cutaneous injection and as sub-lingual application of liquid
compositions according to this invention: Formulation 1 and Formulation 2 at dose of 50 μg and 100 μg/kg respectively

| | Dose | Cₘₐₓ | Tₘₐₓ | t_{1/2} | AUC_{0-∞} | MRT | Relative |
|---|---|---|---|---|---|---|---|
| Formulation | (μg/kg) | (pg/ml) | (Min) | (Min) | (pg/ml)*Min | (Min) | bioavailability |
| Market | 10 | 2287.10 ± 177.53 | 15 | 20.89 ± 12.63 | 71333.79 ± 6137.09 | 39.16 ± 2.27 | — |
| (SC Inj) | | | | | | | |
| Formulation 1 | 50 | 243.87 ± 42.12 | 60 | 21.31 ± 2.46 | 19403.27 ± 2286.94 | 69.25 ± 4.86 | 5.475% |
| Formulation 2 | 100 | 811.49 ± 125.44 | 70 ± 17.32 | 47.34 ± 17.34 | 100126.62 ± 20113.77 | 101.70 ± 16.04 | 14.036% |

*Calculation was done using Non Compartmental analysis in Kinetica 5.0 software
Bioavailability was considered as 100% for 10 μg/kg of subcutaneous injection. Relative bioavailability for 50 μg/kg dose and for 100 μg/kg dose was 5.47% and 14.03% respectively through sub-lingual administration of the monophasic liquid composition of Teriparatide.

1. A water free liquid composition comprising Teriparatide for transmucosal delivery that adheres to mucosa after coming in contact with mucosal surface.

2. The liquid composition according to claim 1 characterized by the same being a monophasic composition comprising Teriparatide.

3. The liquid monophasic composition according to claim 2 comprising a non-aqueous liquid as a carrier, a penetration enhancer/permeation enhancer, stabilizer and a surfactant.

4. The liquid composition according to claim 3 comprising:
a. a liquid carrier selected from the list consisting of Propylene Glycol, Polyethylene Glycol 200, Polyethylene Glycol 400, Glycerol, Ethanol and functionally equivalent liquid Other than above and mixture thereof,
b. a penetration enhancer/permeation enhancer selected form the list consisting of N-acetyl Cysteine, Propylene Glycol, TPGS(d-α-Tocopheryl polyethylene glycol 1000 succinate), Tween 20 and functionally equivalent penetration enhancer/permeation enhancer other than above, and mixture thereof; and
c. a stabilizer selected from the list consisting of, N-acetyl Cysteine, TPGS, or, a functionally equivalent stabilizer other than above, and mixture thereof,
d. a surfactant selected from the list consisting of TPGS, Tween 20 or a functionally equivalent surfactant other than above.

5. The liquid composition according to claim 4 dispensed to a mucosal membrane:
a. of oral/buccal cavity as liquid drops, or
b. of oral/buccal cavity or nasal cavity as an oral or nasal spray or
c. of oral/buccal cavity as an oral film or tablet; or
d. of a part of alimentary canal as incorporated in a capsule or a tablet as an ingredient intended or not intended for site specific delivery of the liquid composition.

6. A system for oral transmucosal delivery of an Active Ingredient through, a liquid composition, the liquid composition transforming itself into a mucoadhesive gel after coming in contact with a mucous membrane;
Wherein,
the liquid composition comprises a monophasic liquid composition of Teriparatide in an inert liquid as carrier, a penetration enhancer/permeation enhancer and a surfactant,
and the monophasic liquid mixture is administered:
a) as drops dispensed directly to the oral mucous membrane, or
b) as a spray directly on the mucous membrane, or
c) through a capsule that, open up on an intended location on a mucous membrane, or
d) as a tablet that disperses on a mucous membrane,
e) as a film that adheres to mucous membrane.

7. The system according to claim 6; wherein:
the Active Ingredient is Teriparatide,
the inert liquid carrier is Polyethylene glycol,
the penetration enhancer/permeation enhancer is N-acetyl cysteine,
the surfactant is, d-α-Tocopheryl polyethylene glycol 1000 succinate (TPGS).
